(19) United States
(12) Patent Application Publication
(10) Pub. No.: US 2012/0179191 A1
(43) Pub. Date: Jul. 12, 2012

(54) INJECTABLE IMPLANTS FOR TISSUE AUGMENTATION AND RESTORATION

(75) Inventor: Mahender Macha, Chelsea, MI (US)

(73) Assignee: HEARTCOR, Chelsea, MI (US)

(21) Appl. No.: 13/424,846

(22) Filed: Mar. 20, 2012

Related U.S. Application Data
(62) Division of application No. 12/612,347, filed on Nov. 4, 2009, now abandoned.
Application No. 12/612,347 is a division of application No. 11/648,971, filed on Jan. 3, 2007, now abandoned.
(60) Provisional application No. 60/756,279, filed on Jan. 3, 2006.

(51) Int. Cl. A61M 29/00

(52) U.S. Cl. 606/191

(57) ABSTRACT

The method and device improves the functioning of dilated body parts and organs by supporting the parts and organs with an injectable and/or implantable biocompatible substance.

### CROSS REFERENCE TO RELATED APPLICATION

This is a divisional of U.S. application Ser. No. 12/612,347 filed Nov. 4, 2009 which is a divisional of U.S. application Ser. No. 11/648,971, filed Jan. 3, 2007, which claims the benefit of U.S. provisional application No. 60/756,279, filed Jan. 3, 2006 which is incorporated by reference as if fully set forth.

### FIELD OF INVENTION

The invention relates generally to the field of tissue augmentation and restoration, and specifically to a system, method, and device for prosthetic injectable implants to augment heart valve tissue and other body tissue.

### BACKGROUND OF THE INVENTION

Mitral regurgitation is a cardiovascular disorder where the heart's mitral valve cannot close properly, causing blood back-flow into the left atrium when the left ventricle contracts. Acute mitral regurgitation may be the result of dysfunction or injury to the valve following a heart attack or infective endocarditis. These conditions may rupture or damage the valve, the papillary muscle, chordae tendineae, or other structures that anchor or support the valve. Damage to these structures may result in the valve leaflet prolapsing, flailing or protruding into the atrium, leaving an opening for the backflow of blood.

This mitral regurgitation is often a complication of dilated cardiomyopathy. In such cases, the mitral regurgitation is considered to be secondary to annular dilatation and altered geometry of the left ventricle. Such “functional” regurgitation results in volume overload of the left atrium during systole, followed by left ventricle and atrium dilatation (remodeling) with further progressive mitral regurgitation and deterioration of ventricular function. This condition is associated with high morbidity and mortality when treated conservatively. (In one study, the one-year survival rate has been reported as being between 30% and 40% for patients with dilated cardiomyopathy with severe mitral regurgitation.)

There are several possible treatments for mitral regurgitation. First, patients with moderate to severe mitral regurgitation typically undergo open heart surgery and excision and replacement of the valve. Second, preservation of the native valve leaflets and repair of the valve by using various techniques includes the implantation of a semi-rigid or rigid annuloplasty ring at the level of the mitral annulus which improves mortality and survival rates. These two treatments are more conventional and generally involve invasive procedures with associated risks of open heart surgery and cardiopulmonary bypass. A third treatment is heart transplantation—a treatment reserved for the sickest patients who might not withstand surgical intervention. During heart transplantation, a surgeon cuts through the patient's breast bone, removes their heart, and sutures a donor's heart in its place. During the transplant operation, the patient's blood circulates through a heart-lung bypass machine to keep the blood oxygen-rich. Following the transplantation, the heart-lung machine is disconnected and the patients blood resumes flowing through the transplanted heart.

The drawbacks with heart transplantation include a limited number of donor hearts available, risks of infection and rejection, and complications associated with surgery that may lead to death. Furthermore, long term survival after heart transplantation is limited by chronic forms of rejection.

In an effort to reduce the risks of surgery, some surgeons perform less invasive mitral valve operations. These less invasive operations may incorporate smaller incisions, thoracoscopic access, or robotic assistance. Cardiopulmonary bypass and arrest of the heart is still required, however, and thus there are significant risks even with these less invasive surgeries.

There has been increasing evidence for the benefits of mitral valve repair even for patients with significant or severe heart failure. Studies have hypothesized that stabilization of the mitral annulus and unloading of the left ventricle may be responsible for the improvement in left ventricle ejection fraction and the reverse remodeling associated with valve repair. Further studies show an increase in left ventricle ejection fraction from 18±5% to 24±10%, and showed an improvement of 25±11% to 34±15% at 2-year follow-up.

Still other studies report improved short-term and mid-term survival after reduction mitral annuloplasty (essentially “down-sizing” the mitral annulus). This modified valve repair appears to demonstrate improved outcomes in patients with dilated cardiomyopathy. Early results showed a 75% 1-year survival.

There are, however, drawbacks in downsizing the annulus through use of a prosthetic ring. For example, the rings are not customized to a specific patient's anatomy because there are only certain sizes available. Moreover, the rings are only available in a rigid, semi-rigid or soft forms that may come loose following surgery. In the event of this loosening, the rings may be reattached or connected in its proper position (in relation to a patient's annulus) in a later surgery. Surgical risks may accordingly increase.

To avoid surgical intervention, there are current attempts to develop percutaneous techniques (i.e., done through a puncture in the skin, typically by a needle and through a very narrow cannula placed in the femoral or jugular vessel) that may achieve plication (i.e., the tightening of stretched or weakened bodily tissues or channels by folding the excess in tucks and suturing) of the annulus of the mitral valve. Such percutaneous approaches to annuloplasty may be accomplished by implanting a plication device in the great cardiac vein/coronary sinus via a known catheter-based delivery device 90 with sheath 92 as shown in FIGS. 1A and 1B. The device 90 has a sheath 92 through which guide mechanisms 94 and an injection tip 96 may travel. The guide mechanism 94 serves as the surgeon's eyes while the injection tip 96 delivers some treatment or serves as a mechanical extensions of the surgeon's fingers.

Having reached the annulus using such a device, the leaflets or a portion of the mitral valve annulus may then be stapled, sutured or the like, thereby effectuating stenosis. One of several drawbacks, however, associated with plication of the annulus through the coronary sinus is that the plication is only normally achieved on one side of the annulus. The effectiveness of this one-sided plication may therefore be less effective to achieving proper remodeling. In addition, such plication devices may obstruct or occlude the venous drainage of the heart, as well as increase the risk of vascular injury or rupture.

There are no FDA approved current interventions that are minimally invasive for the treatment of functional mitral regurgitation. In addition, currently developing technology involves plication or constriction of the annulus through various methods that have significant drawbacks and disadvantages. Thus, there is a need for a minimally invasive yet effective treatment for functional mitral regurgitation.

### SUMMARY OF THE INVENTION

The method described herein injects a biocompatible polymer into or near a damaged or poorly functioning valve, organ, sphincter, or the like. The polymer reshapes the valve or organ in order to improve its function.

The method described herein permits therapeutic intervention that is simpler and easier to apply in valve repair as compared to traditional forms of treatment and less invasive treatment currently employed. Although all forms of valvular heart disease may benefit by the application of the invention, abnormality of the pulmonary valve, aortic valve, or tricuspid valve may also be treated with the method. The method may also be used to improve competence of other pathologically dilated structures such as the gastro-esophageal sphincter, the cervical os, the anal sphincter, or the bladder sphincter. Further, the devices to improve competence of dilated structures and improve valvular function may be delivered through different methods including but not limited to endoscopic delivery, transvenous delivery, laparoscopic surgery, general open surgery, and the like.

While there are implantable prostheses for the treatment of gastroesophageal reflux as well as injectable implants for tissue augmentation and restoration or treating a sphincter, these inventions do not discuss the application of these devices to the treatment of heart disease. The current method and device has not been previously described or implemented. Further, while there are several percutaneous devices in development for the percutaneous plication of the mitral annulus, there are no known devices for the direct augmentation of the mitral annulus. More importantly, none of these use injectable implants.

### BRIEF DESCRIPTION OF THE DRAWINGS

FIGS. 1A and 1B show known catheters.

FIG. 2 shows a partial section through a heart with an implant surrounding and supporting a mitral valve.

FIG. 3 is a section through the body showing the heart and entry point of the catheter.

FIG. 4 shows an injectable implant positioned around the esophaegeal sphincter.

FIG. 5 shows an injectable implant positioned around the bladder sphincter, cervical os, and anal sphincter.

FIG. 6 shows an injectable polymer positioned around the bladder neck.

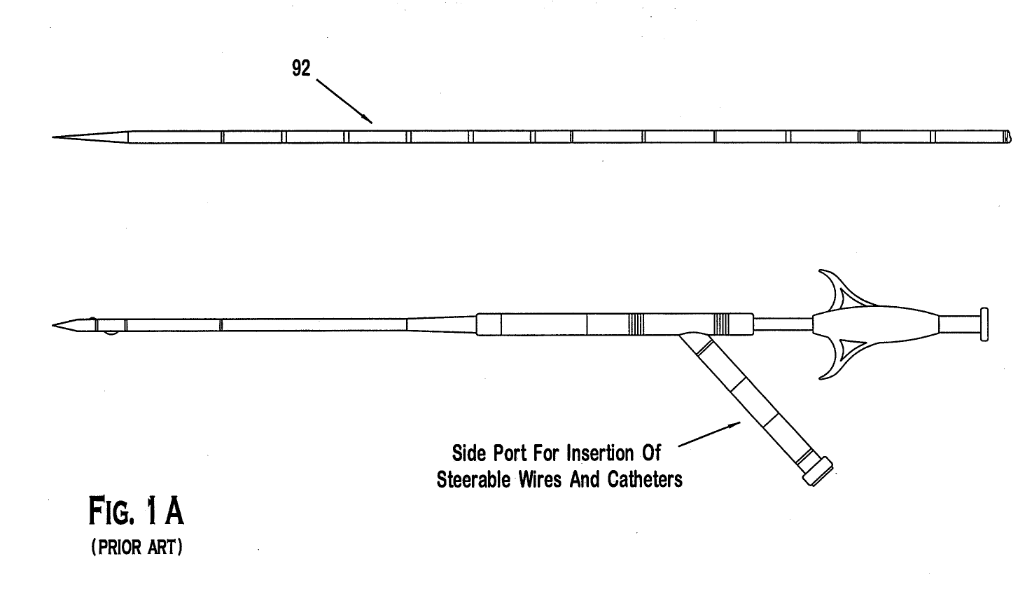

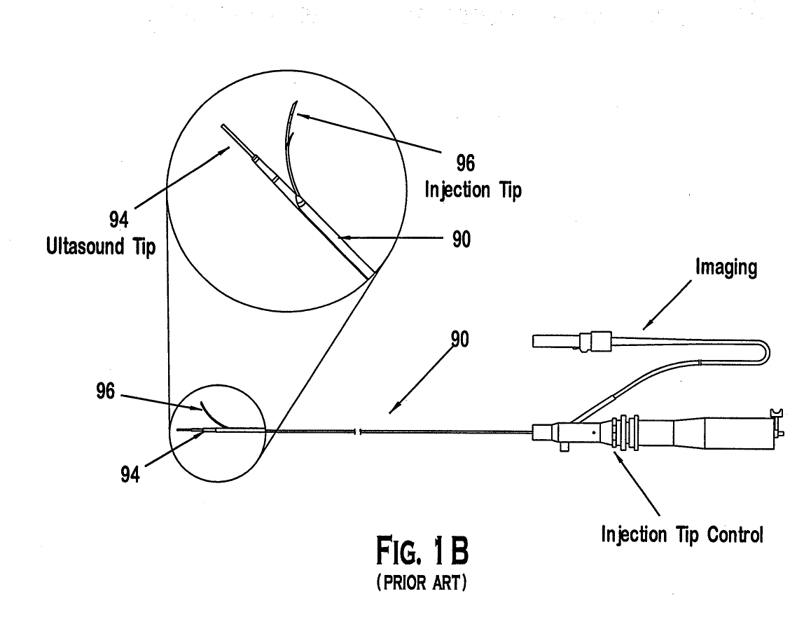

### DETAILED DESCRIPTION OF THE PREFERRED EMBODIMENTS

The method described herein generally comprises the following steps that will be described below in several examples, it being understood that the examples are non-limiting. In applying the biocompatible substance to the body, the following steps are generally common between the examples.

STEP 1. Provide a device that delivers a biocompatible substance. Such a device would typically be capable of sterilely storing and delivering a biocompatible substance. Such devices are well-known and include syringes, needles, and catheters that can be used with guide wires, sheaths (including vascular sheaths), ultrasound guides.

STEP 2. Provide a biocompatible substance. The substance preferably has some combination of the following properties: (1) resists degradation and fluid absorption, (2) maintains flexibility despite improving structural integrity, (3) can be injected as an aqueous solution that solidifies with increased temperature, (4) maintains a solid conformation over a long time period, (5) solidifies at or around body temperature, (6) solidifies at a certain triggering event such as the addition of a catalyst or after a specific amount of time, (7) has antibacterial properties, and (8) is non-toxic, non- mutagenic, and/or non-irritating.

In one embodiment, the substance is a suspension of polymer particles suspended in a collagen solution. In a preferred embodiment, the substance is a suspension of polymethylmethacrylate (PMMA) microspheres suspended in a bovine collagen solution containing buffer, sodium chloride and water. However, the carrier suspension is not so limited and can comprise bovine collagen, human collagen, or any combination thereof. Moreover, the polymer is not limited to PMMA and can be comprised of particles, spheres, grains or fragments of any desired polymer. Preferably, the polymer stimulates tissue fibroblasts to produce a fibrotic capsule that essentially secures the PMMA in place. In addition, it may be preferable that the substance softens over time as the fibrotic response matures and remodels.

In another preferred embodiment, the substance comprises the use of hyaluronic acid gels and the like. Some examples of hyaluronic derivatives include but are not limited to Restylane®, Hylaform® and Rofilan®. In addition, a mixture comprising hyaluronic derivates can also include beads such as Dextran beads and the like.

In yet another embodiment, the polymer material may be comprised of silicone. In another embodiment the substance comprises use of biologic agents such as stem cells or fibroblasts, or adenovirus to augment its efficacy. In another embodiment the substance comprises pharmacologic agents or growth factors that cause a desired effect. In another embodiment, the substance comprises the use of a polymer or beads that are coated with a drug-eluting chemical to improve, augment, or prolong its efficacy.

In yet another embodiment, the biocompatible substance could be used with nanoparticles that could aid in the delivery and targeting particular tissues and absorption of the substance.

In yet another embodiment, the substance comprises a form of gene or cell therapy. Such a therapy could be delivered with an adenovirus, adeno-associated virus, or plasmid. The therapy could, for example, stimulate the production of collagen or enzymes that can alter the extracellular matrix.

STEP 3. Deliver the biocompatible substance to the desired location in the body. Delivery of the biocompatible substance can be done using one of the following methods, perhaps in conjunction with the delivery device: endoscopic delivery, transvascular delivery, laparoscopic surgery, general open surgery, catheter deployment, percutaneous insertion methods, thoracoscopic delivery, etc.

STEP 4. Shape and/or release the substance to form the implant. Releasing the substance to the body part is generally a mechanical process. Shaping the substance is largely application specific. Partially surrounding an annular valve might be preferable in one instance while fully surrounding a valve might be preferable in another. In other applications, the substance may need to be shaped to emulate the body part or otherwise shape or stimulate the body to reestablish normal functioning of the targeted body part.

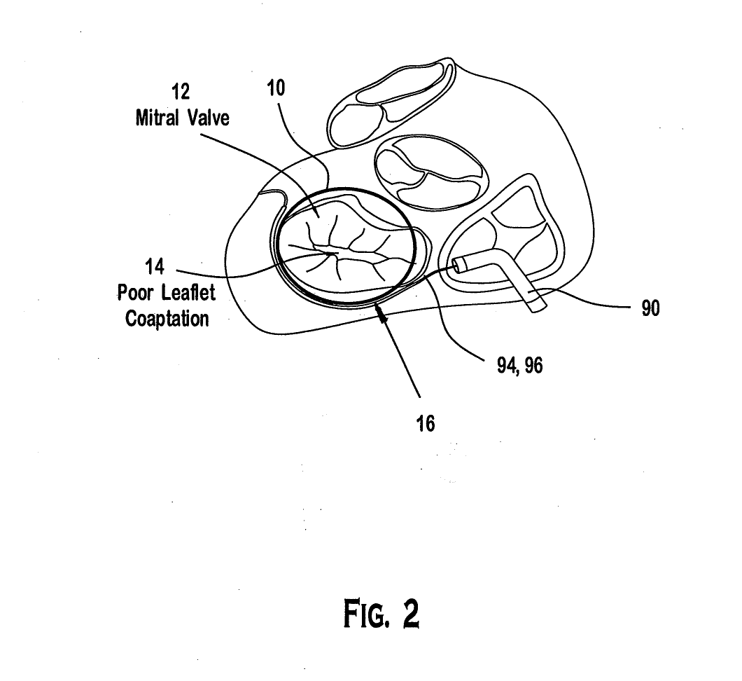

Turning now to some specific examples of using the method, FIGS. 2-6 show the method used with different body parts, in particular but without limitation, the heart, bladder, cervical os, anus, and esophagus.

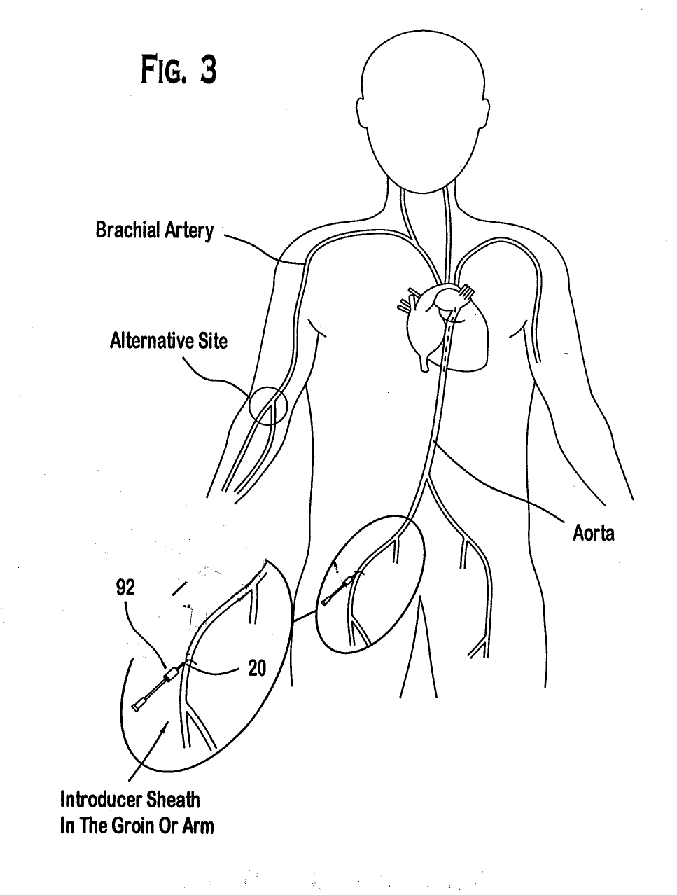

FIGS. 2 and 3 show the method applied to address dilation of a mitral valve in the heart. FIG. 2 shows the mitral valve 12 with leaflets 14 demonstrating malcoaptation, in this case related to dilated cardiomyopathy.

In step 1, a delivery device like a syringe, catheter 90, or other delivery device is provided and in step 2, a biocompatible substance is introduced into this device.

In step 3, the device 90 delivers the biocompatible substance to an area surrounding the mitral valve 12. As shown in FIG. 3, the femoral vein 20 would be accessed by percutaneous needle and a sheath 92 introduced into the vein 20. A catheter deployment system 90 would be introduced through the sheath and guided trans-venously by both fluoroscopy and echocardiography into the coronary sinus. (Such catheter deployment systems are known in the art and need not be discussed in detail.)

As best shown in FIG. 2, the microscopic tip 96 of the catheter 90 would tangentially enter the plane of the posterior mitral annulus across the wall of the coronary sinus and permit circumferential injection of the substance into the heart to form the implant 10. A coronary sinus catheter 16 that could be used in this method is shown in FIG. 2.

In step 4, the substance is shaped and formed. The device needle/catheter permits a surgeon to restore the enlarged mitral annulus to a normal diameter by delivering the biocompatible substance in the region of the annulus to augment and restore its normal diameter, thereby permitting normal coaptation of the mitral valve leaflets 14. As shown in FIG. 2, in one preferred embodiment, the substance is injected into the region of the annulus to form a ring or implant 10 around the annulus. The substance can generally be delivered in the region surrounding and supporting the annulus of the mitral valve 12 to form a constrictive device 10, but it is not so limited. The substance can also be injected directly into the annulus or any portion in close relative proximity to it within the heart.

The substance can augment and restore the normal diameter of the annuluis of the mitral valve 12 in a variety of ways. In one preferred embodiment, the substance stimulates tissue fibrosis. In another embodiment, the substance forms the support necessary to augment and restore the normal diameter of the annulus including but not limited to autologous fat or other soft tissue filler. By augmention and bulking through injection of the substance of these tissues, the mitral valve leaflets 14 will be allowed to coapt in a normal manner and eliminate or reduce mitral valve regurgitation. The augmentation and bulking of the tissue can be performed in a variety of ways such as through a long continuous delivery or through targeted partial injections of the substance. The partial injections of the substance can take place over a predetermined amount of time such as minutes, hours, days, or months. Moreover, the substance can be injected at predetermined locations into or around the annulus. In one embodiment, the injection of the substance is through an open procedure performed with the heart arrested and the patient on cardiopulmonary bypass.

The quantity of substance injected and the precise location of injection along the annulus would be guided by real-time echocardiography which will also assess the correction of the mitral regurgitation. This is unlike any of the other percutaneous techniques in development. Advantages are that it can be performed rapidly and with less risk to the patient. It can be tailored to the precise anatomic requirements of each individual patient to obtain the best possible configuration to optimize leaflet coaptation and eliminate mitral regurgitation. By reshaping and restoring the normal geometry of the mitral annulus using the method described, patients with heart failure may experience prolonged survival.

As generally illustrated in FIGS. 2 and 3, the method permits therapeutic intervention that is simpler and easier to apply in valvular heart disease as compared to traditional forms of treatment discussed above. Although all forms of valvular heart disease may benefit by the application of the invention, mitral regurgitation treatment is probably the most common beneficiary. Abnormality of the pulmonary valve, aortic valve, or tricuspid valve may also be treated with a polymer to improve valvular function.

In use, the present invention can have the same effect as restrictive mitral annuloplasty with a bioprosthetic ring, without the significant drawbacks and disadvantages. The procedure in which the substance is delivered could be performed intra-operatively with an arrested heart, or alternatively, it may be delivered percutaneously via the coronary sinus.

Advantages of this invention over standard heart valve repair techniques involving rigid or semi-rigid annuloplasty rings is the simplicity of application and the feasibility of percutaneous insertion, in addition to the lower risk of death and infection, among others. Further advantages over currently developing technology (one of which involves plication of the annulus) is the ease of application and the reduced tissue injury encountered.

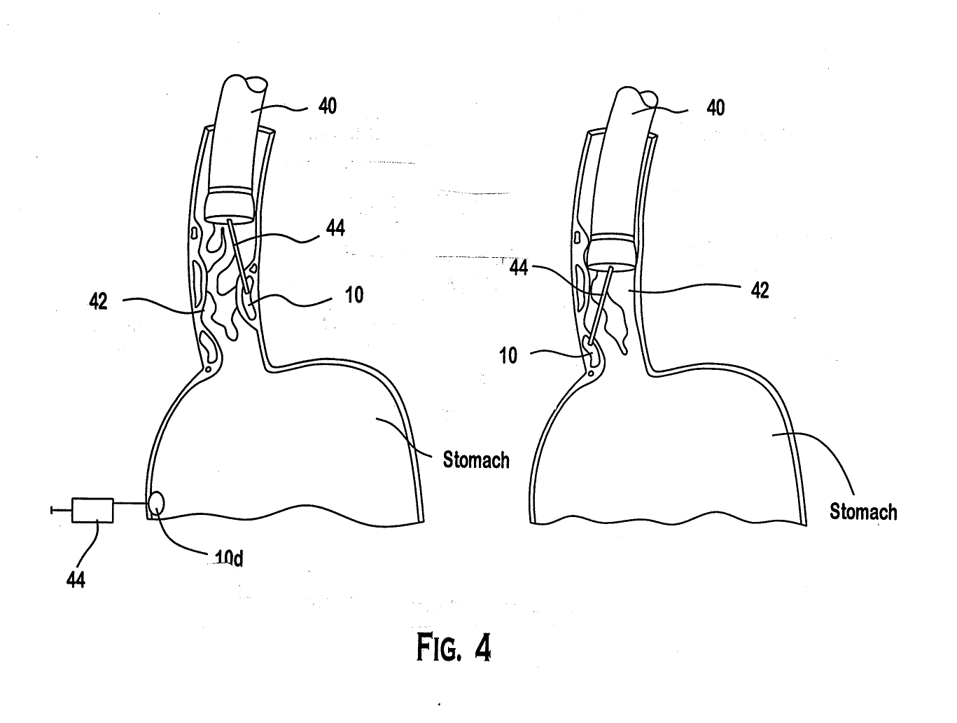

As shown in FIGS. 4-6 the device may also be used to improve competence of other pathologically dilated structures such as the gastro-esophageal sphincter, the cervical os, the anal sphincter, or the bladder sphincter.

In alternate embodiments, the method may be used to treat other diseased or damaged organs in the human body, with the steps described above with respect to the heart being modified to improve substance delivery and shaping. Each of these methods will be discussed in summary below.

1. Gastro-Esophageal Sphincter. FIG. 4.

a. There may be several causes for gastro-esophageal reflux. This may include abnormalities in esophageal or gastric motility, hiatal hernia, diabetes, obesity, pregnancy, or neurological disorders. The most common cause of gastro-esophageal reflux is failure of the lower esophageal sphincter. This muscular tissue opens and closes the lower end of the esophagus, and is vital for maintaining a pressure barrier against contents in the stomach. If this area weakens and loses tone, the lower esophageal sphincter can't close up completely after food enters the stomach. This allows acid from the stomach to back up into the esophagus. This may be related to drugs or dietary factors.

b. The method previously described for the treatment of mitral regurgitation is analogous to the method herein described for the treatment of gastro-esophageal reflux.

c. A standard esophago-gastroscope 40 is inserted under sedation and local analgesia into the oropharynx and advanced to the level of the lower esophageal sphincter 42. A needle 44 is advanced into the submucosal layer of the esophagus and under direct vision, the biocompatible substance is injected circumferentially to form the implant 10. This would effectively augment and remodel the sphincter 42 to reduce incompetence and improve symptoms of gastroesophageal reflux. It may reduce the requirement for more invasive procedures such as surgical fundoplication.

This procedure is tailored to the individual patient pathology and anatomy. The substance may be delivered in larger quantities in areas that require increased bulking and reshaping.

d. The biocompatible substance may have all of the properties previously mentioned. It should resist degradation and absorption, maintain flexibility yet improve structural integrity, be injected as an aqueous solution that solidifies with increased temperature, maintain stability as a solid conformation over a long time period, and have antibacterial properties, and be non-toxic, non-mutagenic, and/or non irritating. Examples of the substance may include PMMA microspheres, human or bovine collagen, hyaluronic acid derivatives, or silicone.

e.

Alternatively, the injection of the substance may also be performed thoracoscopically or laparoscopically.

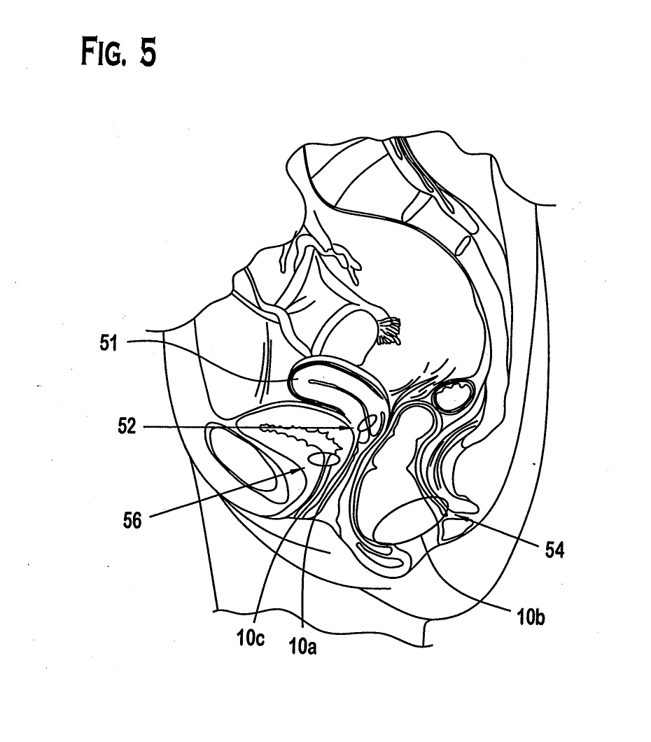

2. Cervical Os. FIG. 5

a. Uterine prolapse results from weakening of the ligaments that support the top of the vagina (called the uterosacral ligaments) and may cause the front and back of the vaginal walls to weaken as well, resulting in prolapse of the uterus 51. Approximately 30-40% of all women experience some type of pelvic organ prolapse. The condition occurs most often in women over the age of 40. It is more common in women who have given birth and in women who have experienced menopause (due to reduced estrogen levels).

For example, as shown in FIG. 5, the cervical os 52 may be augmented endoscopically or with direct surgical exposure to form an implant 10a near the os. This would augment and restore the sphincter mechanism and prevent uterine prolapse, circumventing the need for major surgery.

Procidentia, or rectal prolapse would be prevented by the injection of the substance to form an implant 10b into the submucosa of the anal sphincter 54. This would augment and restore the anal sphincter mechanism and avoid more invasive surgical procedures.

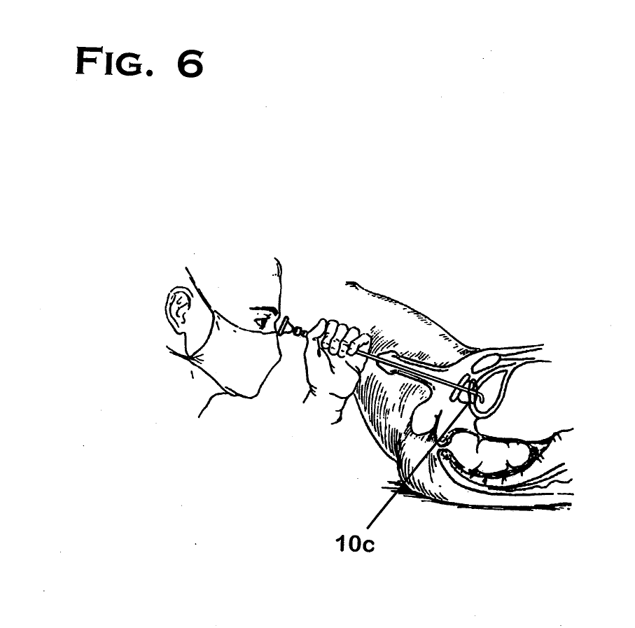

Urinary incontinence may also be effectively reduced by injection of the substance to form an implant 10c into the region of the bladder sphincter 56. This may be delivered trans-urethrally as shown in FIG. 6 (on a man) or by direct surgical exposure.

Additionally as shown in FIG. 4, injection of the substance to form one or more implants 10d in the stomach wall may provide an alternative method for limiting overeating and thereby reducing the risks associated with morbid obesity. The substance can be injected into the stomach wall, the muscle layer of the stomach or any other suitable area of or surrounding the stomach. In one embodiment, the injection of substance can provide a bulking agent to the wall or to the muscle layer of the stomach to reduce the size of the stomach cavity. This may promote the sensation of being full and minimize overeating.

Whereas the present invention has been described in relation to the accompanying drawings, it should be understood that other and further modifications, apart from those shown or suggested herein, may be made within the spirit and scope of the present invention. It is also intended that all matter contained in the foregoing description or shown in the accompanying drawings shall be interpreted as illustrative rather than limiting.

What is claimed is:

1. A method for treating competence of a dilated body part comprising:
providing biocompatible particles; and
delivering the biocompatible particles to the dilated body part using a delivery device for accessing the dilated structure; and
monitoring delivery of the biocompatible polymer particles.

2. The method of claim 1, wherein the biocompatible particles comprise:
polymethlymethacrylate microspheres; and
a bovine collagen solution containing buffer, sodium chloride and water, wherein the polymethlymethacrylate microspheres are suspended in the bovine collagen solution.

3. The method of claim 1, wherein the dilated body part comprises a gastro-esophaegeal sphincter.

4. The method of claim 2, wherein the biocompatible particles are delivered into a submucosal layer of the gastro-esophaegeal sphincter.

5. The method of claim 4, wherein the delivery step comprises injecting the biocompatible particles endoscopically.

6. The method of claim 4, wherein the delivery step comprises surgical access.

7. The method of claim 1, wherein the dilated body part comprises a cervical os.

8. The method of claim 7, wherein the delivery step comprises injecting the biocompatible polymer particles endoscopically.

9. The method of claim 1, wherein the dilated body part comprises an anal sphincter.

10. The method of claim 9, wherein the biocompatible polymer particles are delivered into a submucosa of the anal sphincter.

11. The method of claim 10, wherein the delivery comprises injecting the biocompatible polymer particles endoscopically.

12. The method of claim 1, wherein the dilated body part comprises a bladder sphincter.

13. The method of claim 16, wherein the delivery step comprises delivery of the biocompatible polymer particles trans-urethally.

14. A method for treating cardiovascular disease comprising the steps of:
providing a biocompatible substance; and
delivering the biocompatible substance within or adjacent to a mitral annulus of a human heart, wherein the biocompatible substance within or adjacent to the mitral annulus assists in preventing mitral regurgitation.

15. The method of claim 14, wherein the biocompatible substance stimulates tissue fibrosis to effectuate coaptation of mitral valve leaflets in assisting to prevent mitral regurgitation.

16. The method of claim 14, wherein the biocompatible substance is delivered within or adjacent to the mitral annulus in a liquid state and thereafter hardens, effectuating coaptation of mitral valve leaflets.

17. The method of claim 14, wherein the substance is injected in its liquid state relative to a sub-endothelium along a perimeter of a mitral annulus and thereafter the substance hardens to allow coaptation of mitral valve leaflets.
